(19) United States
(12) Patent Application Publication
(10) Pub. No.: US 2026/0131127 A1
(43) Pub. Date: May 14, 2026

(54) DETACHABLE PASSIVE SEAL FOR MEDICAL DEVICE HAVING ACTIVE SEAL

(71) Applicant: BOSTON SCIENTIFIC SCIMED, INC., MAPLE GROVE, MN (US)

(72) Inventors: Ryan Robert Davis, Plymouth, MN (US); Nicholas Lee Tassoni, Andover, MN (US); Brian Joseph Tischler, Shoreview, MN (US); Jan-Hung Chen, St. Paul, MN (US)

(73) Assignee: BOSTON SCIENTIFIC SCIMED, INC., MAPLE GROVE, MN (US)

(21) Appl. No.: 19/388,374

(22) Filed: Nov. 13, 2025

Related U.S. Application Data
(60) Provisional application No. 63/719,854, filed on Nov. 13, 2024.

(51) Int. Cl. A61M 39/06; A61M 25/00

(52) U.S. Cl. CPC A61M 39/0613; A61M 25/0097; A61M 2039/0633; A61M 2039/0686

(57) ABSTRACT

A medical device assembly includes a medical device and a detachable hemostasis valve. The medical device includes a hub that defines a lumen extending through the hub, and a normally open seal that is disposed within the hub, and an access sheath cap that is adapted to reversibly close the normally open seal. The normally open seal includes an aperture extending through the normally open seal that is coaxial with the lumen. The detachable hemostasis valve includes a valve hub that is adapted to be removably secured to the access sheath cap and a normally closed seal assembly that is disposed within the valve hub.

### CROSS REFERENCE TO RELATED APPLICATIONS

This application claims the benefit of priority of U.S. Provisional Application No. 63/719,854 filed November 13, 2024, the entire disclosure of which is hereby incorporated by reference.

### TECHNICAL FIELD

The disclosure relates generally to medical devices and more particularly to medical devices that include one or more seals or valves.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

The disclosure relates generally to medical devices and more particularly to medical devices that include one or more seals or valves. An example may be found in a medical deice assembly. The medical device assembly includes a medical device and a detachable hemostasis valve. The medical device includes a hub that defines a lumen extending therethrough and a normally open seal that is disposed within the hub. The normally open seal includes an aperture extending therethrough that is coaxial with the lumen. The medical device includes an access sheath cap that is adapted to reversibly close the normally open seal. The detachable hemostasis valve includes a valve hub that is adapted to be removably secured to the access sheath cap and a normally closed seal assembly that is disposed within the valve hub.

Alternatively or additionally, the valve hub may include a graspable portion that is adapted to fit over the access sheath cap, an intermediate portion that defines an annular boss that is adapted to releasably engage an annular groove formed within the hub, an extension portion that extends away from the intermediate portion, and a valve cap that is releasably secured to the extension portion.

Alternatively or additionally, the access sheath cap may include a knurled outer surface and the graspable portion of the valve hub may include an inner surface keyed to the knurled outer surface of the access sheath cap such that rotation of the detachable hemostasis valve causes rotation of the access sheath cap.

Alternatively or additionally, the annular groove formed within the hub may be coaxial with the lumen.

Alternatively or additionally, the valve cap may form an interference fit with the extension portion.

Alternatively or additionally, the normally closed seal assembly may be disposed within the extension portion.

Alternatively or additionally, the normally closed seal assembly may include a first normally closed seal and a second normally closed seal coupled together.

Alternatively or additionally, the first normally closed seal may define a first annular ring that engages a complementary annular surface defined within the extension portion and the second normally closed seal may define a second annular ring that engages a complementary annular surface defined within the valve cap.

Alternatively or additionally, the medical device assembly may further include a distally extending tubular extension that extends through the valve hub and extends through the normally open seal when the valve hub is coupled to the access sheath cap.

Alternatively or additionally, the valve hub may be adapted to be releasably secured to a proximal surface of the access sheath cap.

Alternatively or additionally, the access sheath cap may include an annular ring that is formed within the proximal surface of the access sheath cap and the valve hub may include an annular boss that is adapted to releasably engage the annular ring that is disposed within the proximal surface of the access sheath cap.

Alternatively or additionally, the seal assembly may be entrapped between the access sheath cap and the valve hub.

Alternatively or additionally, the normally open seal assembly may include a first normally closed seal that includes a first annular ring that engages a complementary annular surface that is defined within the proximal surface of the access sheath cap and a second normally closed seal that includes a second annular ring that engages a complementary annular surface that is defined within the valve hub.

Another example may be found in a medical device assembly. The medical device assembly includes a medical device and a detachable hemostasis valve. The medical device includes a hub that defines a lumen extending therethrough and a normally open seal that is disposed within the hub. The normally open seal includes an aperture extending therethrough that is coaxial with the lumen. The medical device includes an access sheath cap that is adapted to reversibly close the normally open seal. The detachable hemostasis valve includes a valve hub that is adapted to be removably secured to the access sheath cap and to extend over the access sheath cap such that rotation of the valve hub causes rotation of the access sheath cap. The detachable hemostasis valve includes a normally closed seal assembly that is disposed within the valve hub.

Alternatively or additionally, the valve hub may include an annular boss that is adapted to releasably engage an annular groove that is formed within the hub, an extension portion that extends proximally from the annular boss, and a valve cap that is releasably secured to the extension portion.

Alternatively or additionally, the annular groove that is formed within the hub may be coaxial with the lumen.

Alternatively or additionally, the normally closed seal assembly may be disposed within the extension portion.

Alternatively or additionally, the normally closed seal assembly may include a first normally closed seal and a second normally closed seal coupled together.

Alternatively or additionally, the first normally closed seal may define a first annular ring that engages a complementary annular surface that is defined within the extension portion and the second normally closed seal may define a second annular ring that engages a complementary annular surface that is defined within the valve cap.

Another example may be found in a detachable hemostasis valve that is adapted to be releasably secured to another medical device having a hub with a normally open seal and a rotatable member adapted to close the normally open seal. The detachable hemostasis valve includes a valve hub that includes a graspable portion that is adapted to fit over the rotatable member, an intermediate portion that defines an annular boss that is adapted to releasably engage an annular groove formed within the hub, and an extension portion that extends away from the intermediate portion. The detachable hemostasis valve includes a valve cap that is releasably secured to the extension portion and a seal assembly that is disposed between the intermediate portion and the valve cap. The seal assembly includes a first normally closed seal that defines a first annular ring that seals against a complementary annular surface defined within the extension portion and a second normally closed seal that defines a second annular ring that seals against a complementary annular surface defined within the valve cap.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 shows selected features of an illustrative steerable catheter;

FIG. 2 is a perspective view of part of an illustrative medical device assembly including a medical device and a detachable hemostasis valve not attached to the medical device;

FIG. 3 is a cross-sectional view taken along the line 3-3 of FIG. 2;

FIG. 4 is a perspective view of part of an illustrative medical device assembly including a medical device and a detachable hemostasis valve attached to the medical device;

FIG. 5 is a cross-sectional view taken along the line 5-5 of FIG. 4;

FIG. 6 is a first exploded perspective view of the illustrative detachable hemostasis valve shown in FIGS. 2-5;

FIG. 7 is a second exploded perspective view of the illustrative detachable hemostasis valve shown in FIGS. 2-5;

FIG. 8 is a schematic cross-sectional view of an illustrative medical device assembly; and

FIG. 9 is a schematic cross-sectional view of an illustrative medical device assembly.

While features of the disclosure are amenable to various modifications and alternative forms, specifics thereof have been shown by way of example in the drawings and will be described in detail. It should be understood, however, that the intention is not to limit features of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified .

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “extent” and/or “maximum extent” may be understood to mean a greatest measurement of a stated or identified dimension, while the term “minimum extent” may be understood to mean a smallest measurement of a stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” or “maximum extent” may be considered a greatest possible dimension measured according to the intended usage. Alternatively, a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

In some instances, a medical device assembly includes a medical device and a detachable hemostasis valve. The medical device includes a hub that defines a lumen extending through the hub, and a normally open seal that is disposed within the hub, and an access sheath cap that is adapted to reversibly close the normally open seal. The normally open seal includes an aperture extending through the normally open seal that is coaxial with the lumen. The detachable hemostasis valve includes a valve hub that is adapted to be removably secured to the access sheath cap and a normally closed seal assembly that is disposed within the valve hub.

In some cases, the valve hub includes a graspable portion that is adapted to fit over the access sheath cap, an intermediate portion that defines an annular boss that is adapted to releasably engage an annular groove formed within the hub, an extension portion that extends away from the intermediate portion, and a valve cap that is releasably secured to the extension portion. In some cases, the access sheath cap may include a knurled outer surface and the graspable portion of the valve hub may include an inner surface keyed to the knurled outer surface of the access sheath cap such that rotation of the detachable hemostasis valve causes rotation of the access sheath cap. In some cases, the annular groove formed within the hub may be coaxial with the lumen. In some cases, the valve cap may form an interference fit with the extension portion. The normally closed seal assembly may be disposed within the extension portion. In some cases, the normally closed seal assembly may include a first normally closed seal and a second normally closed seal coupled together. In some cases, the first normally closed seal may define a first annular ring that engages a complementary annular surface defined within the extension portion and the second normally closed seal may define a second annular ring that engages a complementary annular surface defined within the valve cap.

In some cases, the medical device assembly may further include a distally extending tubular extension that extends through the valve hub and extends through the normally open seal when the valve hub is coupled to the access sheath cap.

In some cases, the valve hub may be adapted to be releasably secured to a proximal surface of the access sheath cap. In some cases, the access sheath cap may include an annular ring formed within the proximal surface of the access sheath cap and the valve hub may include an annular boss that is adapted to releasably engage the annular ring that is disposed within the proximal surface of the access sheath cap. The seal assembly may be entrapped between the access sheath cap and the valve hub, for example. In some cases, the normally open seal assembly may include a first normally closed seal that includes a first annular ring that engages a complementary annular surface defined within the proximal surface of the access sheath cap and a second normally closed seal that includes a second annular ring that engages a complementary annular surface defined within the valve hub.

In some instances, a medical device assembly includes a medical device and a detachable hemostasis valve. The medical device includes a hub that defines a lumen extending therethrough. A normally open seal is disposed within the hub and includes an aperture extending therethrough that is coaxial with the lumen. An access sheath cap is adapted to reversibly close the normally open seal. The detachable hemostasis valve includes a valve hub that is adapted to be removably secured to the access sheath cap and to extend over the access sheath cap such that rotation of the valve hub causes rotation of the access sheath cap. A normally closed seal assembly is disposed within the valve hub.

In some cases, the valve hub may include an annular boss that is adapted to releasably engage an annular groove formed within the hub, an extension portion that extends proximally from the annular boss, and a valve cap that is releasably secured to the extension portion. In some cases, the annular groove formed within the hub may be coaxial with the lumen. The normally closed seal assembly may be disposed within the extension portion. In some cases, the normally closed seal assembly may include a first normally closed seal and a second normally closed seal coupled together. In some cases, the first normally closed seal may define a first annular ring that engages a complementary annular surface defined within the extension portion and the second normally closed seal may define a second annular ring that engages a complementary annular surface defined within the valve cap.

In some instances, a detachable hemostasis valve is adapted to be releasably secured to another medical device having a hub with a normally open seal and a rotatable member adapted to close the normally open seal. The detachable hemostasis valve includes a valve hub that includes a graspable portion that is adapted to fit over the rotatable member, an intermediate portion that defines an annular boss that is adapted to releasably engage an annular groove formed within the hub, and an extension portion that extends away from the intermediate portion. A valve cap is releasably secured to the extension portion and a seal assembly is disposed between the intermediate portion and the valve cap. The seal assembly includes a first normally closed seal that defines a first annular ring that seals against a complementary annular surface defined within the extension portion and a second normally closed seal that defines a second annular ring that seals against a complementary annular surface defined within the valve cap.

In some medical procedures, delivery and/or access sheaths may be routed percutaneously into a body cavity, lumen, and/or treatment site. Navigation through patient vasculature and/or organs may include steering through tortuous anatomy and/or directing a distal end of the delivery and/or access sheath into a body cavity, lumen, and/or treatment site. Examples of medical devices suitable for use in medical procedures, such as but not limited to left atrial appendage closure, aortic valve replacement, mitral valve replacement, septal defect repair, etc., are described herein. Existing medical devices may have certain advantages and/or disadvantages. There is an ongoing need for alternative steerable medical devices for delivering medical implants and/or conducting other treatment procedures. In some cases, a steerable medical device may have a normally open valve or seal such as a Tuohy Borst seal. In some cases, there may be a desire to be able to add the functionality of a normally closed seal to a device having a normally open seal to reduce blood leakage when the normally open seal is open, for example. Having the ability to add a second seal to a device may be applicable to a number of different types of medical devices, but for purposes of illustration will be described herein with respect to a steerable catheter.

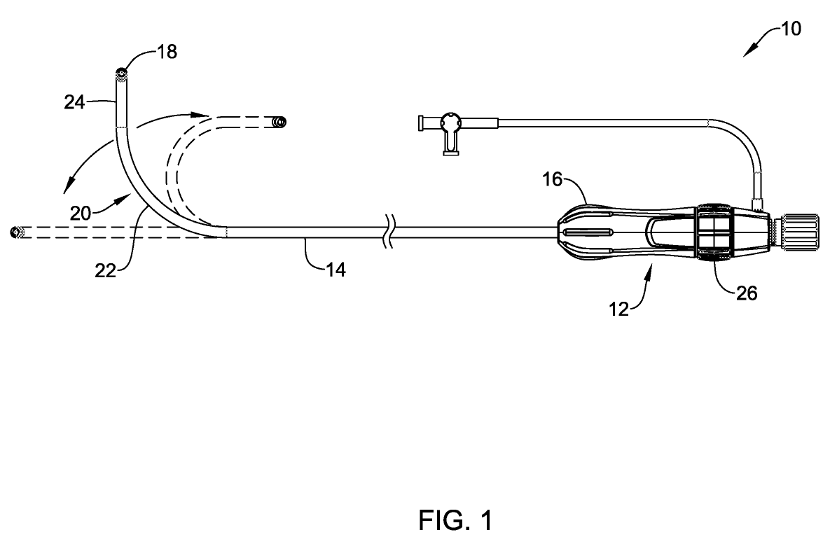

FIG. 1 illustrates selected features of a bi-directional steerable catheter 10. In some cases, the bi-directional steerable catheter 10 may be considered as representing the TruSteer™ steerable catheter available commercially from Boston Scientific. In some instances, the bi-directional steerable catheter 10 may be any one of a variety of catheters, such as an intravascular catheter. Examples of intravascular catheters may include, but are not limited to, balloon catheters, atherectomy catheters, device delivery catheters, drug delivery catheters, diagnostic catheters, and guide catheters. In some cases, the bi-directional steerable catheter 10 may take the form of other suitable guiding, diagnosing, or treating devices (including endoscopic instruments, laparoscopic instruments, etc., and the like) and it may be suitable for use at various locations and/or body lumens within a patient.

The bi-directional steerable catheter 10 may include a handle 12 and an elongate sheath 14 extending distally from the handle 12. In some embodiments, the bi-directional steerable catheter 10 and/or the handle 12 may include a guidewire port, a side port, a fluid flush port, an imaging access port, or other suitable ports, access points, or functional features. The handle 12 may include a handle housing 16. The elongate sheath 14 may extend into and/or through a distal opening in the handle housing 16. In at least some cases, a proximal end of the elongate sheath 14 may be fixedly attached to and/or inside of the handle housing 16. In some cases, a proximal portion of the elongate sheath 14 may include a key element configured to non-rotatably engage one or more lock elements fixedly attached to an inner surface of the handle housing 16 proximal a distal end of the handle housing 16. In some cases, the key element may be bonded to an outer surface of the elongate sheath 14. In some cases, the key element may be integrally formed with the elongate sheath 14. In some cases, the key element may be welded (e.g., heat weld, sonic weld, vibration weld, etc.) to the elongate sheath 14. In some cases, the key element may be melted together with the elongate sheath 14 such that material of the key element is co-mingled with material of the elongate sheath 14 at a molecular level. In some cases, the handle housing 16 may include one or more lock elements fixedly attached to and/or integrally formed with the inner surface of the handle housing 16. In some cases, the one or more lock elements may be formed as ribs or other structural support members configured to increase the rigidity of the handle housing and permit torque transfer between the distal end of the handle housing 16 and the elongate sheath 14. In some cases, the elongate sheath 14 may have a normal or relaxed configuration. The elongate sheath 14 may be self-biased toward, and/or in the absence of any outside forces may return to, the normal or relaxed configuration. Some suitable but non-limiting materials for the handle 12 and/or the handle housing 116 are described below.

In some instances, the elongate sheath 14 may include a soft and/or atraumatic distal tip 18. In some instances, the elongate sheath 14 may include a distal portion 20 having a first curve 22 and a second curve 24, such that the elongate sheath 14 has a preset double curve, in the normal or relaxed configuration. In some instances, the first curve 22 may be preset to curve upwards, as viewed from the side. Other configurations are also contemplated. In some instances, the second curve 24 may be preset to curve to the left, as viewed proximally to distally along the elongate sheath 14. Other configurations are also contemplated. In some instances, the distal portion 20 and/or the first curve 22 may be configured to bend or deflect in a first direction, wherein the distal tip 18 is bent and/or moved towards and/or closer to the handle 12, toward and/or to a deflected configuration, as shown in FIG. 1. In some cases, the distal portion 20 and/or the first curve 22 may be configured to bend or deflect in a second direction opposite the first direction, wherein the distal tip 20 is bent and/or moved away from and/or farther from the handle 12, toward and/or to a straightened configuration, as shown in FIG. 1. In some cases, the elongate sheath 14 may have only a single curve in the normal or relaxed configuration. In some cases, the elongate sheath 14 may be substantially straight in the normal or relaxed configuration. Other configurations, including combinations of those described herein, are also contemplated. In some cases, the handle 12 may include a knurled wheel 26 that may interact with structure inside of the handle housing 16 that may be used to cause the elongate sheath 14 to curve as shown.

Some medical procedures utilize an access device that has a normally closed or passive valve. A passive valve is a valve or seal that is normally closed. No user interaction is needed to close the valve. A passive valve is closed and seals against fluids by default, and only opens when ancillary devices are inserted through the passive valve. Some medical procedures utilize an access device that has a normally open or active valve. An active valve may be opened or closed with user interaction. In some cases, several procedures may be performed on a single part of the body. As an example, a peripheral vein isolation (PVI) procedure may be performed using a FaraDrive™ steerable catheter that employs a passive valve and a FaraWave™ catheter that is advanceable through the FaraDrive™ steerable catheter. A left atrial appendage closure (LAAC) device may subsequently be implanted using a TruSteer™ steerable catheter that has an active valve and a WATCHMAN™ FLX delivery system or a WATCHMAN™ FLX PRO delivery system that is advanceable through the TruSteer™ steerable catheter. Each of these devices are available from Boston Scientific. Currently, to perform a PVI and LAAC procedure at a similar treatment site within a patient, two distinct steerable catheters are utilized because the WATCHMAN™ FLX delivery system and WATCHMAN™ FLX PRO delivery systems are not compatible with the passive valve found in the FaraDrive™ steerable catheter due to the greater insertion forces required to pass an ancillary device through a passive valve versus the open seal utilized in the TruSteer™ steerable catheter.

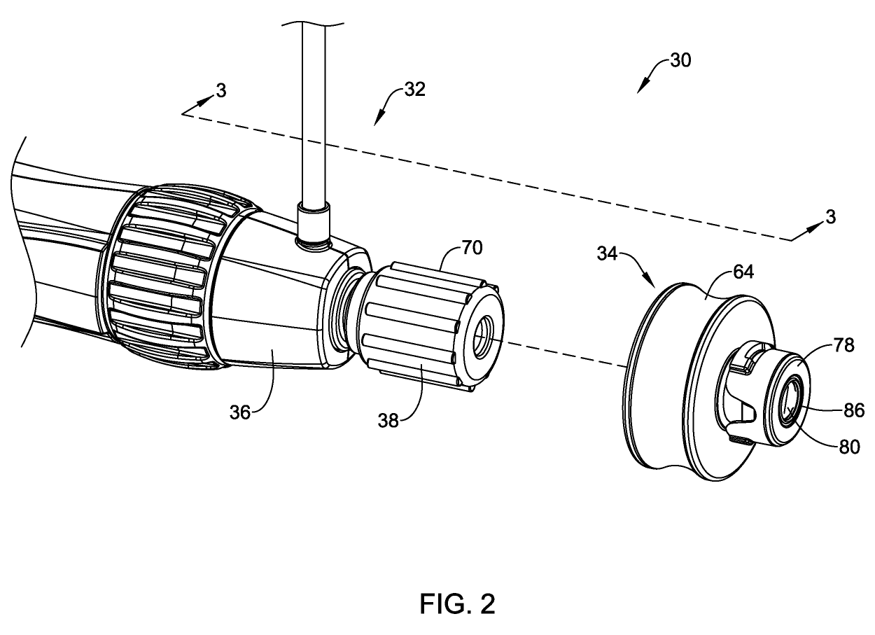

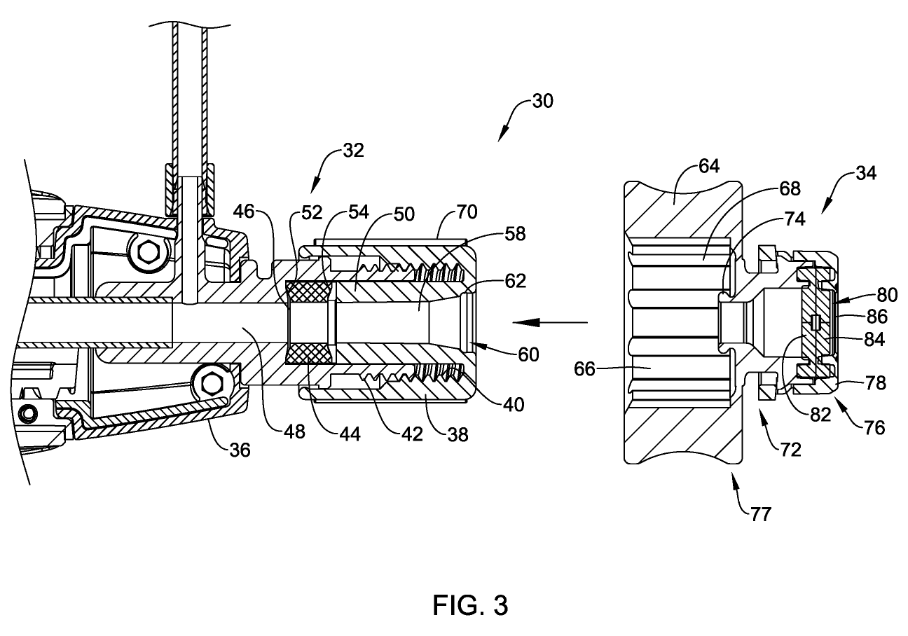

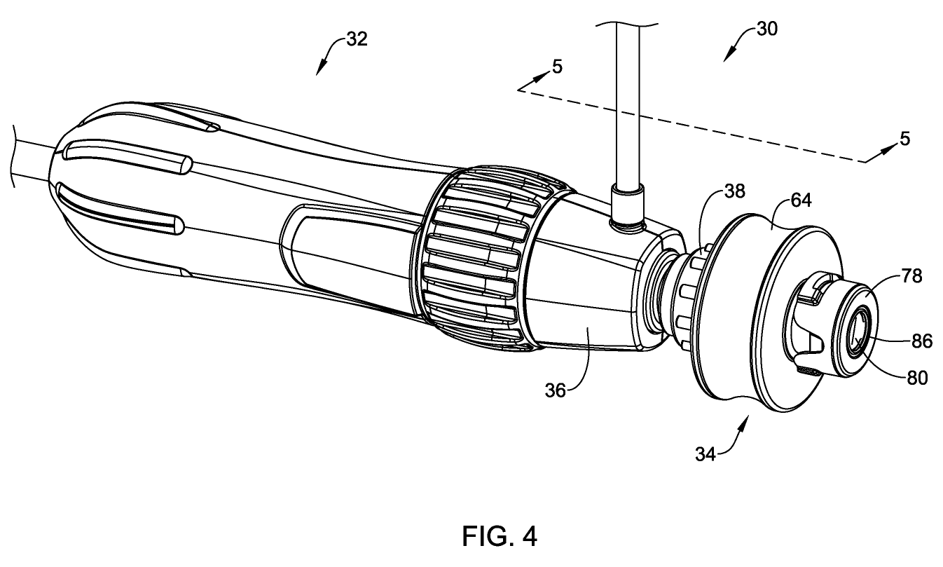

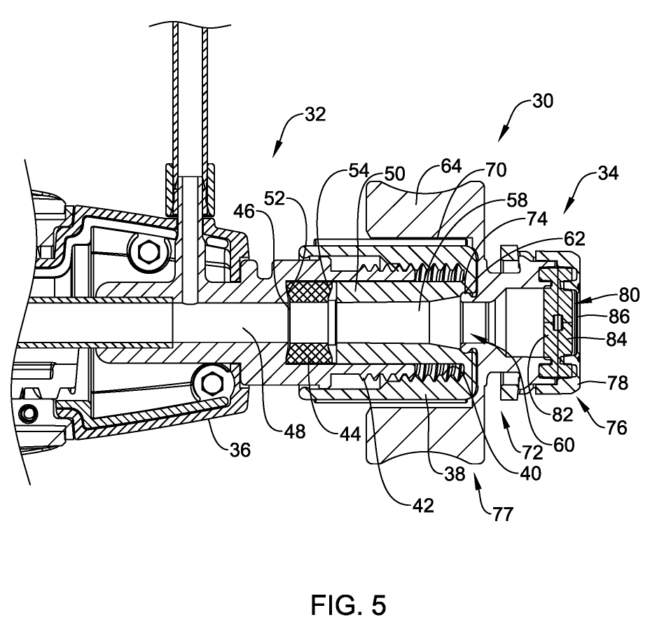

In some cases, a delivery device such as a steerable catheter having an active or normally open seal may be used in combination with a detachable hemostasis valve that provides the functionality of a passive valve or seal when desired, and can be removed when the functionality of the active or normally open seal is desired. FIG. 2 is a perspective view of a portion of an illustrative medical device assembly 30 and FIG. 3 is a cross-sectional view thereof, taken along the line 3-3 of FIG. 2. The portion of the illustrative medical device 30, for example, includes the handle 16 of the bi-directional steerable catheter 10 illustrated in FIG. 1. FIG. 4 is a perspective view of a portion of the illustrative medical device assembly 30 and FIG. 5 is a cross-sectional view thereof, taken along the line 5-5 of FIG. 4. The illustrative medical device assembly 30 includes a medical device 32 and a detachable hemostasis valve 34 that is adapted to be releasably secured to the medical device 32. In FIGS. 2 and 3, the detachable hemostasis valve 34 has not yet been secured to the medical device 32. In FIGS. 4 and 5, the detachable hemostasis valve 34 has been secured to the medical device 32. The detachable hemostasis valve 34 may add the functionality of a passive or normally closed valve to the medical device 32 when that functionality is desired, and the detachable hemostasis valve 34 may be detached when the functionality of an active or normally open valve (as disposed within the medical device 32, as will be discussed) is desired.

The medical device 32 may be considered as representing the bi-directional steerable catheter 10, for example. The medical device 32 includes a hub 36. An access sheath cap 38 is disposed over a proximal region of the hub 36. The access sheath cap 38 includes an inner threaded surface 40 that is adapted to engage a corresponding outer threaded surface 42 on the proximal region of the hub 36. In some cases, the access sheath cap 38 may be moved distally in order for the inner threaded surface 40 to engage the outer threaded surface 42. In some cases, this means that unless the access sheath cap 38 is urged distally, the access sheath cap 38 is able to spin freely without the respective threaded surfaces 40 and 42 engaging each other.

A normally open seal 44 is disposed within the hub 36. In some cases, the normally open seal 44 is a Tuohy Borst valve. The normally open seal 44 may be made of an elastomeric material such as silicone, and may define an aperture 46 that extends axially through the normally open seal 44. In some cases, the hub 36 may be considered as defining a lumen 48 extending through the hub 36. The aperture 46 extending through the normally open seal 44 is coaxial with the lumen 48, such that the aperture 46 becomes part of the lumen 48 when the normally open seal 44 is in the open configuration, as shown. The access sheath cap 38 includes a projection 50 that extends towards the normally open seal 44. In some cases, when the access sheath cap 38 is moved distally and rotated such that the inner threaded surface 40 of the access sheath cap 38 engages the outer threaded surface 42 of the hub 36, the projection 50 will move in a right-to-left direction (with respect to the illustrated orientation), and will push on the normally open seal 44. Because the normally open seal 44 is prevented from moving to the left via an internal wall 52, the projection 50 will cause the normally open seal 44 to become compressed. As the normally open seal 44 is compressed, the aperture 46 extending through the normally open seal 44 will decrease in size, and will eventually close down onto any device being extended through the lumen 48. In some cases, a hard plastic washer 54 may be disposed between the normally open seal 44 and the projection 50. In some cases, the washer 54 may be part of the projection 50.

The access sheath cap 38 includes a lumen 58 that extends through the access sheath cap 38, including the projection 50. The lumen 58 is coaxial with the lumen 48 extending through the hub 36. In some cases, the lumen 58 includes a widened portion 60 that has a larger diameter to make it easier to insert devices through the access sheath cap 38, through the lumen 58 and into the lumen 48. In some cases, the widened portion 60 includes an annular groove 62 that, as will be discussed, may be used to help secure the detachable hemostasis valve 34 to the access sheath cap 38 and hence to the hub 36. In some cases, the annular groove 62 may be considered as being coaxial with the widened portion 60 of the lumen 58.

The detachable hemostasis valve 34 includes a graspable portion 64 that is adapted to fit over the access sheath cap 38. The graspable portion 64 has an inner surface 66 that includes axially-extending recesses 68 that are adapted to engage with corresponding axially-extending ridges 70 that are formed as part of the access sheath cap 38. The ridges 70 form a knurled structure that is easy to grasp and manipulate when the detachable hemostasis valve 34 is not attached. The ridges 70 slide into the recesses 68 when the detachable hemostasis valve 34 is urged into position over the access sheath cap 38. Not only does this allow the detachable hemostasis valve 34 to fit over the access sheath cap 38, but this also allows for an engagement between the detachable hemostasis valve 34 and the access sheath cap 38 that enables a user to rotate the detachable hemostasis valve 34 and concurrently cause the access sheath cap 38 to rotate.

The detachable hemostasis valve 34 includes an intermediate portion 72 that has a smaller diameter than the graspable portion 64. The intermediate portion 72 defines an annular boss 74 that is adapted to engage the corresponding annular groove 62 that is formed within the

widened portion 60 of the lumen 58. An extension portion 76 extends away from the intermediate portion 72. In some cases, the graspable portion 64, the intermediate portion 72 and the extension portion 76 may together be considered as forming a valve hub 77. The valve hub 77 may be an integrally molded component, for example, and may be formed of a rigid polymeric material. A valve cap 78 is releasably secured to the valve hub 77, and in particular is releasably secured to the extension portion 76. The valve cap 78 may form an interference fit with the extension portion 76, for example. A normally closed seal assembly 80 is disposed within the extension portion 76. In some cases, the normally closed seal assembly 80 includes a first normally closed seal 82 and a second normally closed seal 84. In some cases, the valve cap 78 includes an aperture 86 that is coaxial with the lumen 48 and the lumen 58 when the detachable hemostasis valve 34 is positioned on the sheath access cap 38, as shown in FIGS. 4 and 5.

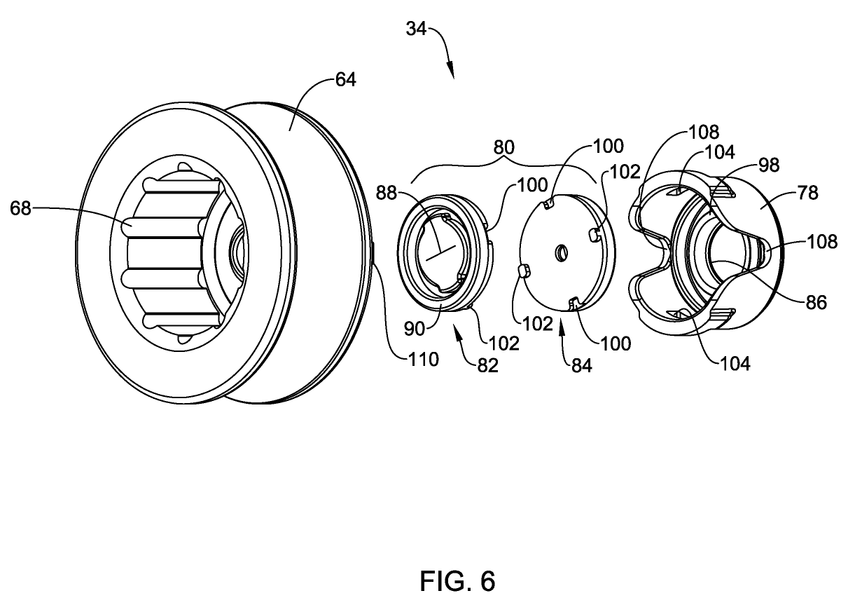

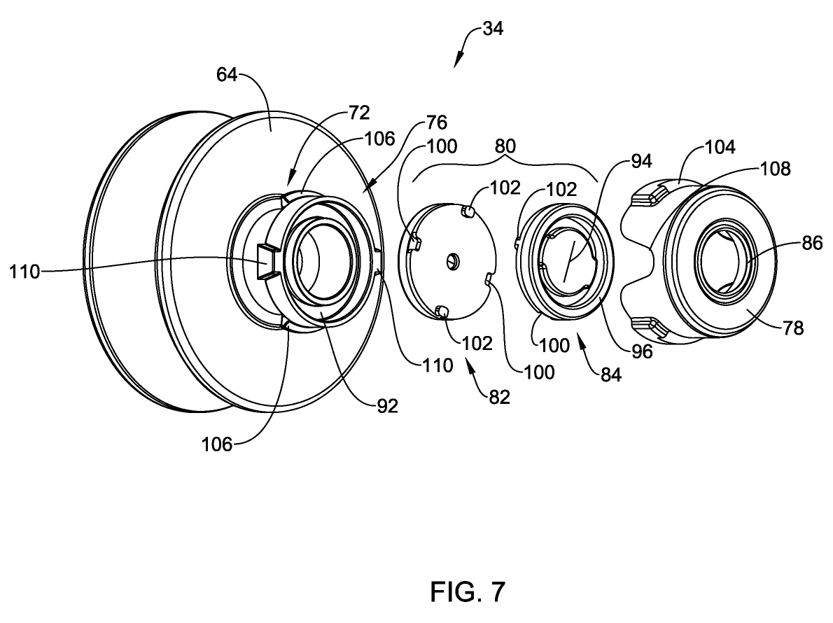

FIGS. 6 and 7 provide additional views of the detachable hemostasis valve 34. FIG. 6 is an exploded perspective view from a first perspective and FIG. 7 is an exploded perspective view from a second perspective in order to reveal additional features of the detachable hemostasis valve 34. The first normally closed seal 82 includes a slit 88 on a side of the first normally closed seal 82 closest to the extension portion 76. The same side of the first normally closed seal 82 includes a first annular ring 90 that engages a complementary annular surface 92 that is defined within the extension portion 76. In some cases, cooperation between the first annular ring 90 and the complementary annular surface 92 helps to prevent fluid leaks around a periphery of the normally closed seal assembly 80. The second normally closed seal 84 includes a slit 94 on a side of the second normally closed seal 84 closest to the valve cap 78. The same side of the second normally closed seal 84 includes a second annular ring 96 that engages a complementary annular surface 98 that is defined within the valve cap 78. In some cases, cooperation between the second annular ring 96 and the complementary annular surface 98 helps to prevent fluid leaks around a periphery of the normally closed seal assembly 80.

In some cases, the slit 88 that is formed within the first normally closed seal 82 may be disposed perpendicularly (i.e., at a ninety degree angle) with respect to the slit 94 that is formed within the second normally closed seal 84. In some cases, the first normally closed seal 82 and the second normally closed seal 84 each include a pair of recesses 100 that are spaced about 180 degrees apart and each include a pair of protrusions 102 that are spaced about 180 degrees apart and each of which are adapted to fit into a corresponding one of the pair of recesses 100 formed within the other seal. The first normally closed seal 82 and the second normally closed seal 84 are arranged such that not only are the slits 88 and 94 rotationally offset about ninety degrees, but so are the recesses 100 and the protrusions 102. In some cases, the valve cap 78 includes recesses 104 that accommodate protrusions 106 that are formed on the extension portion 76 in order to provide a snap-fit between the valve cap 78 and the extension portion 76. In some cases, the valve cap 78 includes cutouts 108 that align with corresponding projections 110 formed on the extension portion 76 to help align the valve cap 78 to the extension portion 76 so that the recesses 104 will align with the protrusions 106. In some embodiments, the devices described herein may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

In some cases, the detachable hemostasis valve 34 may be pre-installed onto the medical device 32, or the detachable hemostasis valve 34 may be secured to the medical device 34 during surgical preparation prior to flushing and degassing the medical device 32 and the detachable hemostasis valve 34. The medical device assembly 30 may be used with the detachable hemostasis valve 34 attached during a PVI treatment. After a PVI treatment, the normally closed seal assembly within the detachable hemostasis valve 34 passively seals the catheter. The normally open seal 44 within the medical device 30 may be closed, and then the detachable hemostasis valve 34 may be detached. Once the detachable hemostasis valve 34 has been detached, the medical device 30 may be used to enable insertion of the WATCHMAN™ delivery system.

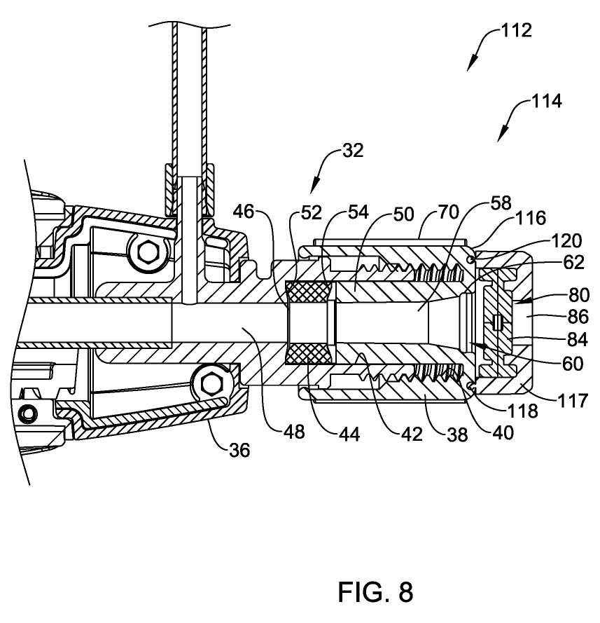

FIG. 8 is a schematic cross-sectional view of an illustrative medical device assembly 112 that includes the medical device 32 and a detachable hemostasis valve 114. The detachable hemostasis valve 114 is adapted to be releasably secured to a proximal surface 116 of the access sheath cap 38. In some cases, the access sheath cap 38 may include an annular ring 118 that is formed within the proximal surface 116 of the access sheath cap 38. The detachable hemostasis valve 114 may include a valve hub 117 that includes an annular boss 120 that is adapted to releasably engage the annular ring 118. The valve hub 117 houses the normally closed seal assembly 80, which may be considered as being entrapped between the access sheath cap 38 and the valve hub 117. In some cases, the detachable hemostasis valve 114 provides for the releasable addition of a normally closed seal with fewer components than the detachable hemostasis valve 34.

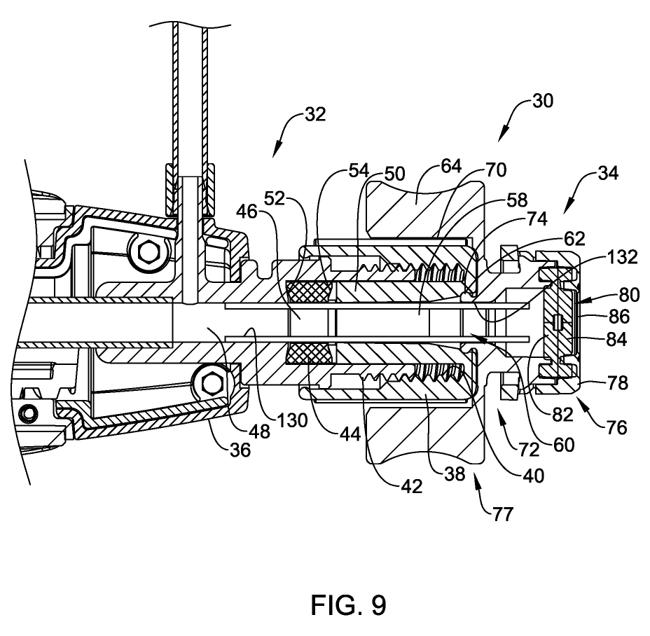

FIG. 9 is similar to FIG. 5, but includes the addition of a distally extending tubular extension 130 that extends distally from the detachable hemostasis valve 34. The distally extending tubular extension 130 may have an outer diameter that is equal to an inner diameter of the lumen 48 and the lumen 58. The distally extending tubular extension 130 may extend through the aperture 46 formed within the normally open seal 44 in order to prevent the normally open seal 44 from being closed, regardless of how the access sheath cap 38 (or the detachable hemostasis valve 34) is actuated or manipulated. In some cases, there may be a desire to eliminate utilization of the normally open seal 44. Possible examples include reduced risk of air entrapment and reduced complexity. The distally extending tubular extension 130 may be attached to any desired part of the valve hub 77. As an example, the distally extending tubular extension 130 may be adhesively secured to an interior surface 132 of the intermediate portion 72.

The materials that can be used for the various components of the bi-directional steerable catheter 10, the detachable hemostasis valve 34, and/or other systems or components disclosed herein and the various elements thereof disclosed herein may include those commonly associated with medical devices. For simplicity purposes, the following discussion makes reference to the sheath, etc. However, this is not intended to limit the devices and methods described herein, as the discussion may be applied to other elements, members, components, or devices disclosed herein, such as, but not limited to, the elongate sheath, the handle, the handle housing, the threaded member(s), the carriage member(s), the steering wire(s), etc. and/or elements or components thereof.

As alluded to herein, within the family of commercially available nickel-titanium or nitinol alloys, is a category designated "linear elastic" or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial "superelastic plateau" or "flag region" in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about –60 degrees Celsius (ºC) to about 120 ºC in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the bi-directional steerable catheter. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the bi-directional steerable catheter to achieve the same result.

In some embodiments, a degree of Magnetic Resonance Imaging (MRI) compatibility is imparted into the bi-directional steerable catheter. For example, the bi-directional steerable catheter and/or components or portions thereof, may be made of a material that does not substantially distort the image and create substantial artifacts (e.g., gaps in the image). Certain ferromagnetic materials, for example, may not be suitable because they may create artifacts in an MRI image. The devices described herein may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the devices described herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the devices described herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure’s scope is, of course, defined in the language in which the appended claims are expressed.

What is claimed is:

1. A medical device assembly, comprising:
a medical device including:
a hub defining a lumen extending therethrough;
a normally open seal disposed within the hub, the normally open seal including an aperture extending therethrough that is coaxial with the lumen;
an access sheath cap adapted to reversibly close the normally open seal; and
a detachable hemostasis valve including:
a valve hub adapted to be removably secured to the access sheath cap; and
a normally closed seal assembly disposed within the valve hub.

2. The medical device assembly of claim 1, wherein the valve hub comprises:
a graspable portion adapted to fit over the access sheath cap;
an intermediate portion defining an annular boss adapted to releasably engage an annular groove formed within the hub;
an extension portion extending away from the intermediate portion; and
a valve cap releasably secured to the extension portion.

3. The medical device assembly of claim 2, wherein:
the access sheath cap comprises a knurled outer surface; and
the graspable portion of the valve hub comprises an inner surface keyed to the knurled outer surface of the access sheath cap such that rotation of the detachable hemostasis valve causes rotation of the access sheath cap.

4. The medical device assembly of claim 2, wherein the annular groove formed within the hub is coaxial with the lumen.

5. The medical device assembly of claim 2, wherein the valve cap forms an interference fit with the extension portion.

6. The medical device assembly of claim 2, wherein the normally closed seal assembly is disposed within the extension portion.

7. The medical device assembly of claim 6, wherein the normally closed seal assembly comprises a first normally closed seal and a second normally closed seal coupled together.

8. The medical device assembly of claim 7, wherein:
the first normally closed seal defines a first annular ring that engages a complementary annular surface defined within the extension portion; and
the second normally closed seal defines a second annular ring that engages a complementary annular surface defined within the valve cap.

9. The medical device assembly of claim 2, further comprising a distally extending tubular extension that extends through the valve hub and extends through the normally open seal when the valve hub is coupled to the access sheath cap.

10. The medical device assembly of claim 1, wherein the valve hub is adapted to be releasably secured to a proximal surface of the access sheath cap.

11. The medical device assembly of claim 10, wherein:
the access sheath cap comprises an annular ring formed within the proximal surface of the access sheath cap; and
the valve hub comprises an annular boss that is adapted to releasably engage the annular ring that is disposed within the proximal surface of the access sheath cap.

12. The medical device assembly of claim 11, wherein the seal assembly is entrapped between the access sheath cap and the valve hub.

13. The medical device assembly of claim 12, wherein the normally open seal assembly comprises:
a first normally closed seal including a first annular ring that engages a complementary annular surface defined within the proximal surface of the access sheath cap; and
a second normally closed seal including a second annular ring that engages a complementary annular surface defined within the valve hub.

14. A medical device assembly, comprising:
a medical device including:
a hub defining a lumen extending therethrough;
a normally open seal disposed within the hub, the normally open seal including an aperture extending therethrough that is coaxial with the lumen;
an access sheath cap adapted to reversibly close the normally open seal; and
a detachable hemostasis valve including:
a valve hub adapted to be removably secured to the access sheath cap, the valve hub adapted to extend over the access sheath cap such that rotation of the valve hub causes rotation of the access sheath cap; and
a normally closed seal assembly disposed within the valve hub.

15. The medical device assembly of claim 14, wherein the valve hub comprises:
an annular boss adapted to releasably engage an annular groove formed within the hub;
an extension portion extending proximally from the annular boss; and
a valve cap releasably secured to the extension portion.

16. The medical device assembly of claim 15, wherein the annular groove formed within the hub is coaxial with the lumen.

17. The medical device assembly of claim 16, wherein the normally closed seal assembly is disposed within the extension portion.

18. The medical device assembly of claim 17, wherein the normally closed seal assembly comprises a first normally closed seal and a second normally closed seal coupled together.

19. The medical device assembly of claim 18, wherein:
the first normally closed seal defines a first annular ring that engages a complementary annular surface defined within the extension portion; and
the second normally closed seal defines a second annular ring that engages a complementary annular surface defined within the valve cap.

20. A detachable hemostasis valve adapted to be releasably secured to another medical device having a hub with a normally open seal and a rotatable member adapted to close the normally open seal, the detachable hemostasis valve comprising:
a valve hub including:
a graspable portion adapted to fit over the rotatable member;
an intermediate portion defining an annular boss adapted to releasably engage an annular groove formed within the hub;
an extension portion extending away from the intermediate portion;
a valve cap releasably secured to the extension portion; and
a seal assembly disposed between the intermediate portion and the valve cap, the seal assembly including:
a first normally closed seal that defines a first annular ring that seals against a complementary annular surface defined within the extension portion; and
a second normally closed seal that defines a second annular ring that seals against a complementary annular surface defined within the valve cap.
